(19) United States
(12) Patent Application Publication
(10) Pub. No.: US 2026/0337483 A1
(43) Pub. Date: Oct. 29, 2026

(54) PERSISTENT LUMINESCENCE NANOPARTICLE FOR THE DETECTION OF H2O2

(71) Applicants: CENTRE NATIONAL DE LA RECHERCHE SCIENTIFIQUE, Paris (FR); UNIVERSITE PARIS CITE, Paris (FR); INSTITUT NATIONAL DE LA SANTE ET DE LA RECHERCHE MEDICALE (INSERM), Paris (FR)

(72) Inventors: Yingshuai LIU, CHONGQING (CN); Jianhua LIU, WEINAN CITY (CN); Cyrille RICHARD, MONTIGNY LE BRETONNEUX (FR); Daniel SCHERMAN, PARIS (FR)

(21) Appl. No.: 19/113,386

(22) PCT Filed: Sep. 19, 2023
(86) PCT No.: PCT/EP2023/075859

§ 371 Date: Mar. 19, 2025

(30) Foreign Application Priority Data
Sep. 19, 2022 (EP) 22315213.3

(51) Int. Cl. C09K 11/62; B82Y 15/00; B82Y 20/00; B82Y 40/00; C09K 11/02; C12Q 1/26; G01N 21/64

(52) U.S. Cl. CPC C09K 11/623; C09K 11/025; C12Q 1/26; G01N 21/6428; B82Y 15/00; B82Y 20/00; B82Y 40/00

(57) ABSTRACT

The present invention relates to a method for the detection of H₂O₂ in a sample, wherein the sample comprises a persistent luminescence nanoparticle.

### FIELD OF THE INVENTION

The present invention relates to a method for the detection of H₂O₂ in a sample comprising a persistent luminescence nanoparticle.

### BACKGROUND OF THE INVENTION

Hydrogen peroxide (H₂O₂) is formed in humans and other animals as a short-lived product in biochemical processes and is toxic to cells. The toxicity is mainly due to oxidation of proteins, membrane lipids and DNA by the peroxide ions. An excessive concentration of H₂O₂ can thus be the cause of certain cancers, cardiovascular diseases or neurodegenerative diseases such as Alzheimer's disease.

Moreover, H₂O₂ can be produced by numerous enzymes, notably the oxidases, in the presence of their substrate. Thus, information on the activity of these enzymes or on the concentration of their substrate can be obtained by measuring the level of H₂O₂ produced by the enzymatic reaction.

As such, H₂O₂, is a biomarker of interest and monitoring its level is of great interest in the field of medical and biological research due to the relationship between its level and diseases and the information about other molecules that can result from the evaluation of H₂O₂.

Several luminescent probes have been developed for the optical detection of H₂O₂ such as colorimetric sensors in which an organic molecule (tetramethylbenzidine, TMB) can be oxidized by H₂O₂ in the presence of an enzyme (horseradish peroxidase, HRP) and H₂SO₄ to form a colored product, whose absorbance depends on the concentration of H₂O₂ (Bally et al. (1989) J. Clin. Chem. Clin. Biochem., 27:791-796). However, sensitivity of detection of those methods is low.

To improve the sensitivity of the detection, fluorescent probes have been explored using fluorogenic enzyme substrates, such as for example commercially available Amplex Red, which become fluorescent in presence of H₂O₂ and peroxidase. Although the sensitivity of organic fluorescent dyes is very high, one drawback is their poor photostability.

Optical nanomaterials have better photostability, so optical sensors for H₂O₂ based on nanomaterials were also developed in recently years, such as gold nanoparticles (Shiang et al. (2009) Chemical communications, 3437-3439), silver nanoparticles (Teodoro et al. (2019) Carbohydrate Polymers, 212:235-241), quantum dots, carbon dots, up-conversion nanoparticles, lanthanide-based nanoparticles (Casanova et al. (2009), Nat Nanotechnol, 4: 581-585).

However, using such optical probes requires constant excitation and undesirable auto-fluorescence and elevated background signals result in poor signal-to-noise ratio reducing the sensitivity of the assay.

Thus, there is a need for a simple, economic, and efficient method for detecting H₂O₂ which is also applicable to the measurement of low levels of various molecules able to produce H₂O₂ such as an enzyme or its substrate.

### SUMMARY OF THE INVENTION

The present invention arises from the unexpected finding by the inventors that the intensity of the signal of a persistent luminescence nanoparticle in a sample, in particular a zinc gallate persistent luminescence nanoparticle, is amplified in the presence of H₂O₂. More, particularly, the inventors have found that the intensity of the signal of the persistent luminescence nanoparticle varies proportionally with the amount of H₂O₂ in the sample.

The inventors have also found that the method for detecting H₂O₂ is applicable to the detection of low levels of molecules able to produce H₂O₂, such as glucose, when acting as substrate of particular enzymes.

The inventors have also found that the method for detecting H₂O₂ is applicable to the detection of H₂O₂ producing particular enzymes, such as glucose oxidase, when a particular substrate is added to the reaction medium.

The inventors have also found that the method for detecting H₂O₂ is applicable to the detection of analyte, such as an antigen, which can be associated with a reaction inducing the production of H₂O₂.

Thus, the present invention relates to a method for the detection of H₂O₂ in a sample, wherein the sample comprises a persistent luminescence nanoparticle.

The present invention also relates to the detection and/or quantification of any compound which chemical modification leads to H₂O₂ production in situ, or any enzyme which reaction leads to H₂O₂ production in situ, or any analyte which can be associated with a reaction inducing the production of H₂O₂ in situ.

The present invention also relates to the detection of H₂O₂ producing enzymes, and of substrate leading to H₂O₂ production upon enzymatic reaction in a sample.

The present invention also relates to the use of a persistent luminescence nanoparticle for the quantification and/or detection of H₂O₂ in a sample.

The present invention also relates to the use of a persistent luminescence nanoparticle for the quantification and/or detection of H₂O₂ producing enzymes, or of substrate leading to H₂O₂ production upon enzymatic reaction in a sample, or of analytes which can be associated with a reaction inducing the production of H₂O₂.

The present invention also relates to a kit for detecting H₂O₂ in a sample comprising a persistent luminescence nanoparticle and an enzyme catalysing a reaction producing H₂O₂.

The present invention also relates to a method for the preparation of a persistent luminescence nanoparticle comprising a step of calcination at a temperature lower than 750 °C.

### DETAILED DESCRIPTION OF THE INVENTION

### Definition

As intended herein, the word “comprising” is synonymous to “including” or “containing”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

It must be noted that as used herein and in the appended claims, the singular forms “a”, “an”, and “the” include plural references unless the context clearly dictates otherwise. As well, the terms “a”, “an”, “one or more” and “at least one” can be used interchangeably herein.

In the context of this application, the term “about” defines a range of plus or minus 10% of the cited value.

### Persistent Luminescence Nanoparticle

The expression “persistent luminescence nanoparticle” as used herein is well known from the person skilled in the art and relates to nanoparticle with persistent luminescence properties. As intended herein, persistent luminescence nanoparticles emit light after being excited by an excitation source. The persistence time are evaluated by following the intensity of luminescence as a function of time after excitation. The expression “persistent luminescence nanomaterial” is applied to nanomaterials having a luminescence of at least 1 second up to several hours or even several days.

Preferably, the persistent luminescence nanoparticle according to the invention emits light after being excited by an excitation source.

The excitation of the persistent luminescence nanoparticle according to the invention can be carried out by any suitable excitation source well known by the person skilled in the art. The person skilled in the art can easily determine the type of excitation source to be used in the frame of the present invention.

Preferably, the excitation of the persistent luminescence nanoparticle according to the invention is carried out by wavelengths of the ultraviolet (UV), visible or infrared (IR), by radiation X, chemical reactions (chemiluminescence), enzymatic reactions (bioluminescence), electrical excitations (electroluminescence) or mechanical excitations (triboluminescence). More preferably, the excitation of the persistent luminescence nanoparticle according to the invention is carried out at a wavelength between 100 and 1700 nm, preferably between 100 nm and 1400 nm or between 100 nm and 800 nm.

Preferably, the excitation source according to the invention comprises a UV light source, a visible light source or an infrared light source. Any material well known by the person skilled in the art and suitable for exciting the persistent luminescence nanoparticle according to the invention can be used as excitation source. By way of example, it is possible to use a UV lamp, a light emitting diode or a halogen.

Preferably, the persistent luminescence nanoparticle according to the invention is excited with a UV lamp at 254 nm.

The excitation time of the persistent luminescence nanoparticle according to the invention can be easily adjusted by the person skilled in the art. Preferably, the persistent luminescence nanoparticle according to the invention is excited by an excitation source for at least 0.01 second. Preferably, the persistent luminescence nanoparticle is excited for a period of 1 second to 10 hours, for example for 1 second to 8 hours, 1 second to 6 hours, 1 second to 4 hours, 1 second to 2 hours, or 1 second to 1 hour. More preferably, the persistent luminescence nanoparticle is excited by an excitation source for 1 second to 30 minutes, for example for 1 second to 20 minutes, 1 second to 15 minutes, 1 second to 10 minutes, 1 second to 8 minutes, 1 second to 7 minutes, 1 second to 6 minutes, 1 second to 5 minutes, 1 second to 4 minutes, 1 second to 3 minutes, 1 second to 2 minutes or 1 second to 1 minute. More preferably, the persistent luminescence nanoparticle is excited for a period comprised between 10 seconds and 10 minutes, even more preferably between 40 seconds and 10 minutes.

Preferably, the persistent luminescence nanoparticle according to the invention emits light at any wavelength known to a person skilled in the art. By way of example, the persistent luminescence nanoparticle according to the invention can emit light in the visible region of the electromagnetic spectrum, in the UV region of the electromagnetic spectrum, in the IR region of the electromagnetic spectrum, in the near IR (NIR) region of the electromagnetic spectrum, in the near X-ray region of the electromagnetic spectrum, or in multiple regions of the electromagnetic spectrum.

Preferably, the persistent luminescence nanoparticle according to the invention emits light at a wavelength between 100 nm and 1700 nm, for example between 400 nm and 1700 nm, or between 400 nm and 1400 nm.

Preferably, the persistent luminescence nanoparticle according to the invention emits light for at least 1 second, for example for at least 1 minute, at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes after excitation of the persistent luminescence nanoparticle has ceased.

Preferably, the persistent luminescence nanoparticle according to the invention emits light after the excitation has ceased for 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 12 hours, 13 hours, 14 hours, 15 hours, 16 hours, 17 hours, 18 hours, 19 hours or 20 hours.

More preferably, the persistent luminescence nanoparticle according to the invention emits light for 1 minute to 20 hours after the excitation has ceased, more preferably, for 1 hour to 15 hours, 1 hour to 12 hours, 1 hour to 10 hours, 1 hour to 5 hours, or 1 hour to 3 hours.

Preferably, the persistent luminescence nanoparticle according to the invention has a size comprised between 1 nm and 1000 nm, for example between 1 nm and 900 nm, 1 nm and 800 nm, 1 nm and 700 nm, 1 nm and 600 nm, 1 nm and 500 nm, 1 nm and 400 nm, 1 nm and 300 nm, 1 nm and 200 nm, 1 nm and 100 nm. More preferably, the persistent luminescence nanoparticle according to the invention has a size comprised between 10 nm and 200 nm, 10 nm and 150 nm, 10 nm and 100 nm, 20 nm and 80 nm, 20 nm and 50 nm, even more preferably, of about 30 nm.

The size of the persistent luminescence nanoparticle according to the invention can be determined by any conventional method suitable for measuring the size of nanoparticles. By way of example of method which can be used for determining the size of the persistent luminescence nanoparticle according to the invention it is possible to cite transmission electron microscopy (TEM).

The persistent luminescence nanoparticle according to the invention can be selected from any persistent luminescence nanoparticle known by the person skilled in the art. By way of example of persistent luminescence nanoparticle it is possible to cite the LuPO₄, LaPO₄, YPO₄, ScPO₄, GdPO₄, CaTiO₃, BaTiO₃, SrTiO₃, NaNbO₃, KNbO₃, SrSc₂O₃, CaSiO₃, Sr3SiO5, SrSi2O2N2, BaZrSi3O9, SrSiO₃, BaSiO₃, Zn2SiO4, Ca2Si5O8, Sr₂SnO₄, Ca₂SnO4, Ba₂SnO₄, Gd₂O₂CO₃, metal oxide nanoparticles such as NiO, ZnO, MnO₂, CaO, CuO, CeO₂, Ag₂O, Fe₂O₃, TiO₂, Y₂O₃, La₂O₃, Yb₂O₃, HO₂O₃, Tm₂O₃, Lu₂O₃, Tb₄O₇, Nd₂O₃, Sm₂O₃, Gd₂O₃, Er₂O₃, Pr₆O₁₁, Eu₂O₃, Dy₂O₃, MgO, SrO, BaO, ZrO₂, FeO, V₂O₃, V₂O₅, Mn₂O₃, Al₂O₃, SiO₂, Co₃O₄, and combinations thereof; aluminate nanoparticles such as CoAl₂O₄, NiAl₂O₄, CuAl₂O₄, MgAl₂O₄, ZnAl₂O₄, SrAl₂O₄, CaAl₂O₄, BaAl₂O₄, LaAlO₃, GdAlO₃, EuAlO₃, ErAlO₃, NdAlO₃, HoAlO₃, TmAlO₃, SmAlO₃, TbAlO₃, YAlO₃, YbAlO₃, Al₂FeO₄, LaAlO₃, LiAlO₂, AlCeO₃, BaMg2Al2N4, ZnMnAl2O4, CaTiO3, oxysulfide nanoparticles including yttrium-based compounds such as yttrium oxide sulfides (Y2O₂S, etc.), germanate nanoparticles such as MgGeO₃, CaGeO₃, ZnGeO₃, Zn2GeO4, Ca2GeO4, Li2MgGeO4, FeGeO₃, CuGeO₃, LiScGeO4, Na2CaGe2O6, CaZnGe2O6, Ca2Ga2GeO7, Sr2MgGe2O7, Sr2Ga2GeO7, Bi₄Ge₃O₁₂, Ca₂Ge₇O₁₆, SrGe₄O₉, BaGe₄O₉, HfGeO₄, ThGeO₄, GeO₃Pb, and combinations thereof, zinc gallate nanoparticle such as ZnGa₂O₄, CaGa2O4, LiGa₅O₈, Lu3Ga5O12, Gd3Ga5O12, Y3Ga5O12, MgGa₂O₄, CuGa₂O₄, ZnGa₂AlO₄, LaGaO₃, LaMgGa₁₁O₁₉, LaZnAl_{1.5}Ga_{9.5}O₁₉, SrGa₁₂O₁₉, GaTaO4, InGaO4, ScGaO4, gallogermanates such as ZnGa₂GeO₄, Zn₃Ga₂Ge₂O₁₀, Zn₃Ga₂GeO₈, Zn(Ga₁-xAlx)₂O₄, Mg₃Ga₂GeO₈, Mg3Y2Ge3O12, Mg4Ga8Ge2O20, La₃Ga₅GeO₁₄, La₃GaGe₅O₁₆, garnets such as Y₃Al₂Ga₃O₁₂, Lu3Al2Ga3O12, Zn₃Al₂Ge₃O₁₂, Y₃Ga₅O₁₂, Y₃Al₅O₁₂, Gd₃, Ga₅O₁₂, Gd₃Al₂Ga₃O₁₂, Gd₃Zn₂GaGe₂O₁₂, Ca₃Ga₂Ge₃O₁₂, titanate nanoparticles Na2TiO3, Gd2TiO3, Ca3Ti2O7, including M-TiO₂ wherein M is magnesium or zinc, sulfide nanoparticle such as zinc sulfide (ZnS), calcium sulfide (CaS) and strontium sulfide (SrS), nickelate nanoparticles, silicate nanoparticles such as ZnMgSi₂O₆, CaMgSi₂O₆ and MgSiO₃, Ca₂MgSi2O7, aluminosilicates nanoparticles such as BaSrAlSi5O2N, Ca2Al2SiO7, CaAlSiN3, SrAlSiN3, phosphates nanoparticles, vanadates nanoparticles, niobate nanoparticles and combinations thereof.

Preferably, the persistent luminescence nanoparticle according to the invention does not comprise Germanium (Ge).

Preferably, the persistent luminescence nanoparticle according to the invention does not comprise ZnGaGeO, in particular Zn_{1.2}Ga_{1.6}Ge_{0.2}O₄, more particularly Zn_{1.2}Ga_{1.6}Ge_{0.2}O₄:Cr³⁺. Preferably also, the persistent luminescence nanoparticle according to the invention is not ZnGaGeO-based, in particular Zn_{1.2}Ga_{1.6}Ge_{0.2}O₄-based, more particularly Zn_{1.2}Ga_{1.6}Ge_{0.2}O₄:Cr³⁺-based.

Preferably, the persistent luminescence nanoparticle according to the invention is doped with one or more chemical element. By way of example of chemical element which can be used, it is possible to cite manganese, chromium, bismuth, magnesium, scandium, titanium, vanadium, tin, iron, cobalt, nickel, copper, zinc, zirconium, niobium, molybdenum, technetium, ruthenium, rhodium, palladium, silver, cadmium, hafnium, tantalum, tungsten, rhenium, osmium, iridium, platinum, gold, mercury, a rare-earth element such as yttrium, lanthanum, cerium, praseodymium, neodymium, promethium, samarium, europium, gadolinium, terbium, dysprosium, holmium, erbium, thulium, ytterbium, lutetium, scandium, and combinations thereof. Preferably, the rare earth element is found in the trivalent form thereof (Ce³⁺, Dy³⁺, Nd³⁺, Ho³⁺, Er³⁺, etc.) except for europium, samarium and ytterbium, which may also be found in the divalent form thereof (Eu²⁺, Sm²⁺ and Yb²⁺).

Preferably, doping the persistent luminescence nanoparticle with one or more chemical element replaces a small amount of the atoms in the persistent luminescence nanoparticle with the chemical element which is used as a dopant.

Preferably, the persistent luminescence nanoparticle is doped with between 0.01 mol % and 50 mol % of one or more chemical element for example with between 0.01 mol % and 45 mol %, with between 0.01 mol % and 40 mol %, with between 0.01 mol % and 35 mol %, with between 0.01 mol % and 30 mol %, with between 0.01 mol % and 25 mol %, with between 0.125 mol % and 20 mol %, with between 0.125 mol % and 15 mol %, with between 0.125 mol % and 10 mol %, with between 0.125 mol % and 5 mol %, with between 0.125 mol % and 2 mol %, with between 0.125 mol % and 1.5 mol %, with between 0.125 mol % and 1 mol %. More preferably, the amount of chemical element in the persistent luminescence nanoparticle is 0.25 mol %.

Preferably, the persistent luminescence nanoparticle according to the invention is a zinc gallate nanoparticle.

More preferably, the persistent luminescence nanoparticle according to the invention is a ZnGa₂O₄ persistent luminescence nanoparticle.

Preferably, the persistent luminescence nanoparticle according to the invention is a ZnGa₂O₄ persistent luminescence nanoparticle doped with chromium ions, or chromium ions co-doped with other cations. Preferably, the other cations are selected from the group consisting of a manganese ion (Mn²⁺), a tin ion (Sn⁴⁺), a gadolinium ion (Gd³⁺), a ferrous ion (Fe³⁺), a nickel ion (Ni²⁺), a europium ion (Eu³⁺) and a samarium ion (Sm³⁺).

More preferably, the persistent luminescence nanoparticle according to the invention is a ZnGa₂O₄ persistent luminescence nanoparticle doped with chromium ions, or chromium ions co-doped with a manganese ion (Mn²⁺) or a tin ion (Sn⁴⁺). Preferably, the amount of chromium ions, or chromium ions co-doped with other cations in the ZnGa₂O₄ persistent luminescence nanoparticle according to the invention is comprised between 0.10 mol % and 2 mol %, for example between 0.125 mol % and 0.5 mol %, or between 0.25 mol % and 0.5 mol %.

Preferably, the persistent luminescence nanoparticle according to the invention is selected from the group consisting of a ZnGa₂O₄: Cr³⁺, also known as chromium-doped zinc gallate nanoparticles or ZGO nanoparticle, a ZnGa₂O₄: Cr³⁺, Mn²⁺ and a ZnGa₂O₄: Cr³⁺, Sn⁴⁺.

Preferably, the persistent luminescence nanoparticle according to the invention is dispersed in a medium, more preferably an aqueous solution. By way of example of medium which can be used for dispersing the persistent luminescence nanoparticle according to the invention it is possible to cite H₂O, NaCl, PBS, TAPS, Tris, Tricine, Bicine, TAPSO, TES, MES, HEPES, MOPS, Bis-tris methane, ADA, Bis-tris propane, ACES, MOPSO, TEA, HEPBS, CHES, PIPES and more complex medium such as serum and human serum.

The persistent luminescence nanoparticles according to the invention can be stored in a dried form or dispersed in an aqueous solution at room temperature or at a lower temperature such as 4 °C. The persistent luminescence nanoparticles according to the invention can also be stored in a solid form frozen at −80 °C.

Preferably, the concentration of the persistent luminescence nanoparticle according to the invention in the solution, in particular the aqueous solution, is comprised between 0.015 mg/ml to 1 mg/ml, more preferably between 0.015 mg/ml to 0.05 mg/ml.

Preferably, the amount of the persistent luminescence nanoparticle needed for the detection of H₂O₂ in a sample is comprised between 0.2 μg and 250 μg, for example between 0.5 μg and 200 μg, 0.5 μg and 100 μg, 0.5 μg and 90 μg, 0.5 μg and 80μg, 0.5 μg and 70μg, 0.5 μg and 60 μg, 0.5 μg and 50 μg, 0.5 μg and 40 μg, 0.5 μg and 30 μg, 0.5 μg and 25 μg, 0.5 μg and 15 μg, 0.5 μg and 10 μg, 0.5 μg and 9 μg, 0.5 μg and 8 μg, 0.5 μg and 7 μg, 0.5 μg and 6 μg, 0.5 μg and 5 μg, 0.5 μg and 4 μg, 0.5 μg and 3 μg, 0.5 μg and 2 μg, 0.5 μg and 1 μg.

Preferably, the amount of the persistent luminescence nanoparticle needed for the detection of H₂O₂ in a sample is about 1 μg.

The suspensions comprising the persistent luminescence nanoparticle according to the invention may further comprise one or more buffer or pH adjuster.

The persistent luminescence nanoparticle according to the invention can be synthesized by any method well known by the person skilled in the art.

By way of example, the ZGO nanoparticle is well known to the person skilled in the art and can be synthesized according to the following method:
dissolving gallium oxide in concentrated HNO₃ (35%);
heating the mixture to 150 °C;
dissolving Cr(NO₃)₃.9H₂O and Zn(NO₃)₂.6H₂O in deionized water to form a solution which is mixed with the solution of Ga(NO₃)₃;
adding ammonia solution to adjust the pH to 7.5;
heating to 120 °C;
washing the resulting product.

In an embodiment, the method for the preparation of the persistent luminescence nanoparticle according to the invention comprises a step of calcination or heating of the nanoparticle.

Preferably, the calcination of the persistent luminescence nanoparticle according to the invention is performed at a temperature comprised between 200 °C and 1200 °C. More preferably, the calcination of the persistent luminescence nanoparticle according to the invention is performed at a temperature equal or lower than 750 °C, even more preferably, at a temperature comprised between 500 °C and 750 °C.

According to an embodiment of the present invention, the persistent luminescence nanoparticle is obtained by a method comprising a step of calcination of the persistent luminescence nanoparticle at 500 °C.

According to an embodiment of the present invention, the persistent luminescence nanoparticle is obtained by a method comprising a step of calcination of the persistent luminescence nanoparticle at 750 °C.

Advantageously, the luminescence of the persistent luminescence nanoparticle is amplified by a calcination of the persistent luminescence nanoparticle at a temperature equal or lower than 750 °C, preferably between 500 °C and 750 °C.

Advantageously, the lowest concentration of molecule that can be detected (LOD) in a sample according to the invention is improved when the persistent luminescence nanoparticle is prepared with a calcination step at a temperature equal or lower than 750 °C, preferably between 500 °C and 750 °C.

The persistent luminescence nanoparticle according to the invention can be used in non-functionalized, coated, functionalized and/or encapsulated form.

Preferably, the coating, functionalization, and encapsulation of the persistent luminescence nanoparticle may be carried out according to standard methods known to those skilled in the art.

According to an embodiment of the present description, the persistent luminescence nanoparticle is functionalized by coating with a chemical molecule and/or by grafting a biological or chemical substance or a ligand. Preferably, the biological or chemical substance or ligand according to the invention is selected from the group consisting of an antibody, a protein, such as avidin, a biotin, a peptide, a phosphate, a carboxylate, a polymer, and a free amine group etc. More preferably, the persistent luminescence nanoparticle is functionalized with a biological or chemical substance selected from the group consisting of a polymer, a biotin, a protein, in particular avidin, and an antibody.

By way of example, when the persistent luminescence nanoparticle according to the invention is functionalized with an avidin, the avidin is preferably bound to the biotinylated persistent luminescence nanoparticle.

By way of example also, when the persistent luminescence nanoparticle according to the invention is functionalized with an antibody, the antibody is preferably bound to an avidin which is bound to the biotinylated persistent luminescence nanoparticle.

The coating methods are well known to the person skilled in the art. For example, the coating may be performed by bonding with molecules carrying phosphonic acid, or carboxylate groups, or by means of the heteroprecipitation of silica, aminosilane, or triethoxyaminopropylsilane. The coating can be performed by surface precipitation of triethoxyaminopropylsilane, by polymerization of monomers or polymers to the surface of the nanoparticles, or by hydration in the presence of lipids.

The method for grafting (or coupling) a ligand, a biological or a chemical substance are well known to the person skilled in the art. It generally consists of coupling by means of a covalent bond, affinity, passive or forced adsorption. In the case of coupling by means of a covalent bond, the nanoparticles carry chemical groups capable of reacting with another chemical group carried by the substance to be grafted to form a covalent bond. By way of examples of chemical groups liable to be present on the surface of the nanoparticles, it is possible to cite, but without being limited thereto, hydroxyl, carboxyl, amino, aldehyde and epoxy groups.

Passive or forced adsorption coupling is known to those skilled in the art. It is possible to use for example BSA-biotin (Bovine Serum Albumin) or glucose oxidase (GOD).

It is also possible to use interaction by means of affinity, which is generally applied by two partners of a high-affinity bonding pair such as in particular, but without being limited thereto, (poly)carbohydrate/lectin pairs, biotin or biotinylated/avidin or streptavidin compounds, receptor/specific ligand or hapten/antibody, etc.

The grafting of the coated nanoparticles may also be carried out either directly or using spacer arms also referred to using the terms “linker” or “spacer”.

Preferably, the persistent luminescence nanoparticles according to the invention comprise a coating. More preferably, the persistent luminescence nanoparticle according to the invention comprises a polymer coating. Preferably, the persistent luminescence nanoparticles according to the invention do not comprise a quencher-based, in particular a MnO₂-based, coating. Preferably, where the persistent luminescence nanoparticles according to the invention comprise a coating, the coating does not comprise a quencher, in particular MnO₂.

The polymer can be chosen among any polymer well known to the person skilled in the art.

Preferably, the polymer coating according to the invention is selected from the group consisting of polyethylene glycol, ethylene glycol, polyvinylpyrrolidone, polypropylene glycol, polystyrene, polylactic acid, polyvinyl methyl ether, polyisoprene, polyacrylonitrile, polyacrylate, polyvinyl ethyl ether, polyvinyl alcohol, polyvinyl esters such as polyvinyl acetate and poly(vinyl cinnamate), polyvinylpyrrolidone, polyacrylics and polyacrylates such as polyhydroxypropyl acrylate, poly(N-2-hydroxypropyl)methacrylamide (pHPMA), polymethacrylate, poly (methyl methacrylate), polyacrylic acid, poly(poly(ethylene glycol methacrylate)-stat-dimethyl(methacryoyloxy) phosphonic acid), polyesters such as polyglycolide, polyglycolic acid, polylactic acid, polycaprolactone, polyhydroxyalkanoate, polyhydroxybutyrate, polyethylene adipate, polybutylene succinate, poly(3-hydroxybutyrate-co-3-hydroxyvalerate), polyethylene terephthalate, polybutylene terephthalate, polytrimethylene terephthalate, polyethylene naphthalate, methyl cellulose, hydroxyethyl cellulose, hydroxypropyl methyl cellulose, hydroxypropyl cellulose, ethyl hydroxyethyl cellulose polypropylene, polyethylene and combinations thereof.

More preferably, the polymer used for coating the persistent nanoparticle according to the invention is selected from the group consisting of polyethylene glycol, poly(N-2-hydroxypropyl)methacrylamide (pHPMA) and a derivative of polyethylene glycol linked to phosphonic acid.

The persistent luminescence nanoparticle can be coated by any method well known to the person skilled in the art. By way of example, chromium-doped zinc gallate nanoparticles coated with polyethylene glycol (ZGO-PEG NPs) can be prepared as followed:
hydroxylated chromium-doped zinc gallate nanoparticles (ZGO—OH) are first converted to ZGO—NH₂nanoparticles by adding 3-aminopropyl-triethoxysilane (APTES) to a suspension of ZGO—OH nanoparticles in DMF;
the reaction mixture is sonicated and stirred a room temperature;
particles are then washed from unreacted APTES;
ZGO-PEG NPs are obtained by reacting ZGO—NH₂ with NHS-PEG;
ZGO-PEG NPs are then washed with DMF and water.

Alternatively, ZGO-pHPMA can be obtained by reacting ZGO—NH₂ with semitelechelic p(HPMA)-TT.

Alternatively, ZGO-pPEG can be obtained by reacting ZGO—OH with phosphonic acid PEG derivatives (pPEG) in water.

Alternatively, ZGO-PEG-biotin can be obtained by reacting ZGO—NH₂ with NHS-PEG-biotin.

The ZGO-PEG-biotin can then be coated with avidin.

The ZGO-PEG-biotin can also bind a biotinylated antibody or a biotinylated glucose oxidase after having been coated with avidin. Avidin can be used either to coat the ZGO-PEG biotin or to coat the biotinylated antibody on the plate.

### Method for the Detection of H₂O₂

Preferably, the presence of H₂O₂ in a sample comprising a persistent luminescence nanoparticle according to the invention induces a variation of the intensity of the luminescence. Preferably, the variation of luminescence corresponds to an increase of the signal. In particular, the increase in the intensity of the luminescence induced by H₂O₂ is not due to an H₂O₂-induced dequenching of the persistent luminescence nanoparticle according to the invention, or the luminescence of the persistent luminescence nanoparticle is not quenched by an H₂O₂-sensitive quencher bound to the nanoparticle or coating the nanoparticle. More particularly, the increase in the intensity of the luminescence induced by H₂O₂ is not due to an H₂O₂-induced reduction of an MnO₂-based coating of the persistent luminescence nanoparticle according to the invention. More preferably, the increase of the signal varies proportionally with the amount of H₂O₂ in the sample.

The persistent luminescence nanoparticle according to the invention is particularly adapted for the detection and/or quantification of H₂O₂ in a sample.

The method according to the invention is also particularly adapted to the detection and quantification of any compound which chemical modification leads to H₂O₂ production in situ, or any enzyme whose reaction leads to H₂O₂ production in situ, or any analyte which can be associated with a reaction inducing the production of H₂O₂ in situ.

As intended herein, the term “analyte” refers to any compound in the sample whose amount or concentration can be deduced from the production of H₂O₂, such as an enzyme, a substrate of an enzyme, a molecule, an antigen, an antibody, etc.

As intended herein the expression “in situ” means in the reaction medium.

Preferably, the enzyme able to produce H₂O₂ in situ by an enzymatic reaction, preferably in the presence of its substrate, is selected from the group consisting of superoxide dismutase, and oxidase. More preferably, the enzyme able to produce H₂O₂ in situ is an oxidase. Preferably, the oxidase able to produce H₂O₂ in situ is selected from the group consisting of glucose oxidase, lactate oxidase, alcohol oxidase, cholesterol oxidase, galactose oxidase, oxalate oxidase, glyoxal oxidase, glycollate oxidase, monoamine oxidase and uricase. The enzyme able to produce H₂O₂ in situ according to the invention can be fixed on an antibody or a protein, such as avidin. The antibody can be chosen among any antibody well known by the person skilled in the art. By way of example, the antibody can be chosen among monoclonal antibodies and polyclonal antibodies.

Preferably, the analyte which can be associated with a reaction inducing the production of H₂O₂ in situ is selected from the group consisting of an antigen, an antibody, glucose, lactic acid and uric acid.

Advantageously, the detection of H₂O₂ produced by an enzymatic reaction in a sample comprising a persistent luminescence nanoparticle according to the invention can give information on the activity and/or concentration of an enzyme or its substrate. Thus, the method for detecting H₂O₂ according to the invention is applicable to the measurement of low levels of various molecules, in particular enzymes when a particular substrate is added to the reaction medium and substrates of particular enzymes.

Advantageously also, the detection of H₂O₂, by way of example produced by an enzymatic reaction, in a sample comprising a persistent luminescence nanoparticle according to the invention can give information on the activity and/or concentration of an antigen.

In an embodiment, the present invention relates to the detection in a sample of H₂O₂ wherein the sample comprises a persistent luminescence nanoparticle according to the invention.

In an embodiment, the present invention relates to the detection and/or quantification of H₂O₂ produced in situ by a molecule.

In an embodiment, the present invention relates to a method for the detection and/or quantification of H₂O₂ produced in situ by an enzyme.

In an embodiment, the present invention relates to a method for the detection and/or quantification of H₂O₂ produced in situ by a molecule acting as substrate of particular enzyme.

The present invention also relates to a method for the detection and/or quantification of H₂O₂ produced in situ by an enzyme fixed on an antibody or a protein, such as avidin.

In an embodiment, the present invention relates to a method for the detection and/or quantification of a molecule selected from the group consisting of glucose, lactic acid, and uric acid, in a sample, in the presence of an enzyme, in particular an oxidase.

Preferably, the method according to the invention for the detection and/or quantification of H₂O₂ in a sample is implemented as an ELISA test, in particular a direct ELISA, a sandwich ELISA, or as test strip.

Advantageously, the detection of the signal can be done when the parasitic auto-fluorescence produced by fluorescent compounds present in the biological media and/or by constituents present in the supports used (plates, tubes, strips) has disappeared. Preferably, the detection of the luminescence of the persistent luminescence nanoparticle according to the invention starts at least 5 seconds, preferably at least 10 seconds after the excitation has ceased.

In an embodiment, the method for the detection of H₂O₂ according to the invention comprises the steps of:
exciting the persistent luminescence nanoparticle in a sample comprising H₂O₂, preferably in a wavelength comprised between 100 nm and 1700 nm; and
detecting the signal emitted by the persistent luminescence nanoparticle, preferably by measuring the signal emitted by the persistent luminescence nanoparticles by optical imaging.

In an embodiment, the method for the detection of H₂O₂ according to the invention comprises the steps of:
exciting the persistent luminescence nanoparticle in a sample comprising an enzyme such as glucose oxidase, which may be bound to an antibody or a protein through avidin/biotin interaction, preferably in a wavelength comprised between 100 nm and 1700 nm;
detecting the persistent luminescence nanoparticle, preferably by measuring the persistent luminescence nanoparticles by optical imaging. This embodiment is suitable for example for the dosage of molecule such as glucose, able to produce H₂O₂ through an enzymatic reaction in the presence a particular enzyme such as glucose oxidase. This embodiment is also suitable for the detection of an antigen when the enzyme is bound to an antibody.

Preferably, the two previous embodiments are performed with non-functionalized nanoparticles. Preferably, when the detection is implemented in the form of an ELISA test, the same amount of persistent luminescence nanoparticle is introduced in each well, there is no need to wash the plate before measuring the signal. Preferably, the signal emitted by the persistent luminescence nanoparticle increases with increasing amount of H₂O₂. Preferably, the enhancement, defined as the signal of the persistent luminescence nanoparticle in the presence of H₂O₂ divided by the signal of the persistent luminescence nanoparticle without H₂O₂, is proportional to the amount of H₂O₂.

In an embodiment, the method for the detection of H₂O₂ according to the invention comprises the steps of:
exciting the persistent luminescence nanoparticles functionalized with an antibody, a biotin or an avidin, in a sample comprising H₂O₂, preferably in a wavelength comprised between 100 nm and 1700 nm;
detecting the persistent luminescence nanoparticle, preferably by measuring the persistent luminescence nanoparticles by optical imaging.

By way of example, when the detection is implemented as an ELISA test, the method for the detection of H₂O₂ according to the invention can comprise the steps of:
preparing the surface of the plate with an antibody;
applying the sample comprising the antigen to the plate;
adding an antibody labelled with avidin;
adding functionalized persistent luminescence nanoparticles, preferably the functionalized persistent luminescence nanoparticles is selected from biotinylated-persistent luminescence nanoparticles, such as biotinylated-ZGO nanoparticles;
washing the plate
adding H₂O₂;
measuring the signal of the persistent luminescence nanoparticle, preferably by a photon-counting device.

By way of example, when the detection is implemented as an ELISA test, the method for the detection of H₂O₂ according to the invention can comprise the steps of:
preparing the surface of the plate with an antibody;
applying the sample comprising the antigen to the plate;
adding an antibody labelled with biotin;
adding functionalized persistent luminescence nanoparticles, preferably the functionalized persistent luminescence nanoparticles is selected from biotinylated-persistent luminescence nanoparticles coated with avidin, such as avidin-ZGO nanoparticles;
washing the plate
adding H₂O₂;
measuring the signal of the persistent luminescence nanoparticle, preferably by a photon-counting device.

By way of example also, the method for the detection of H₂O₂ according to the invention can comprise the steps of:
preparing the surface of the plate with an antibody;
applying the sample comprising the antigen to the plate; adding the antibody functionalized-persistent luminescence nanoparticles, antibody functionalized-ZGO nanoparticles;
washing the plate
adding H₂O₂;
measuring the signal of the ZGO nanoparticle, preferably by a photon-counting device.

Preferably, the plate is washed before the addition of H₂O₂.

Preferably, the signal of the persistent luminescence nanoparticles is amplified by H₂O₂ and is dependent of the amount of nanoparticles retained in each well, which is in turn dependent on the quantity of antigen present.

Preferably, in the case of functionalized nanoparticles, the detection limit is lowered upon addition of H₂O₂.

The person skilled in the art will know how to adapt the protocol for an ELISA test to a protocol for a strip test.

Alternatively, H₂O₂ can be produced in situ by way of example when an antibody fixed on an avidin carrying an enzyme, or when avidin carrying and enzyme is bound to the persistent luminescence nanoparticles and the substrate of the enzyme is added to the medium. By way of example, H₂O₂ can be produced in situ using an avidin carrying a glucose oxidase (avidin-GOD) and by adding glucose in the medium.

The detection of the persistent luminescence nanoparticle can be performed by any suitable optical imaging material, such as camera and/or detector, well known by the person skilled in the art. By way of example of material which can be used, it is possible to cite a photodetector such as photomultiplier tube, a charge coupled device (CCD, ICCD), or a photon-counting device.

### Use of the Persistent Luminescence Nanoparticle

The persistent luminescence nanoparticle according to the present invention is particularly adapted for a use in the field of biological analysis and/or immunological analysis.

The persistent luminescence nanoparticle according to the present invention is particularly adapted in theranostic.

The persistent luminescence nanoparticle according to the present invention can be used as a probe for optical detection of H₂O₂.

Preferably, the persistent luminescence nanoparticle according to the present invention is used for the quantification and/or detection of H₂O₂ in a sample.

Preferably, the persistent luminescence nanoparticle according to the present invention is used for the quantification and/or detection of any molecule able to produce H₂O₂ in situ by an enzymatic reaction. The persistent luminescence nanoparticle according to the present invention is used for the quantification and/or detection of an enzyme, a substrate whose enzymatic consumption produces H₂O₂, or an antigen.

Preferably, the persistent luminescence nanoparticle according to the present invention is used as an in vitro diagnostic agent.

Preferably, the persistent luminescence nanoparticle according to the present invention is used in a composition for in vitro diagnostic.

### Kit

The kit according to the present invention may further comprise instructions for the detection of H₂O₂.

The kit according to the present invention may further comprise tubes or vials containing uncoated or coated nanoparticles, either in a dried formed, or dispersed in a solvent or a buffer, to be stored at room temperature or in dry ice. The tubes can contain the nanoparticles (coated or not) at different concentrations.

The kit according to the present invention may further comprise tubes of H₂O₂ at different concentrations.

The kit according to the present invention may further comprise vials of glucose, glucose oxidase and avidin.

The kit according to the present invention may further comprise instructions for using the nanoparticles.

The present invention is further illustrated with reference to the following examples. These examples are provided for illustrating the present invention and should not be construed as limiting the scope and spirit of the present invention.

### DESCRIPTION OF THE FIGURES

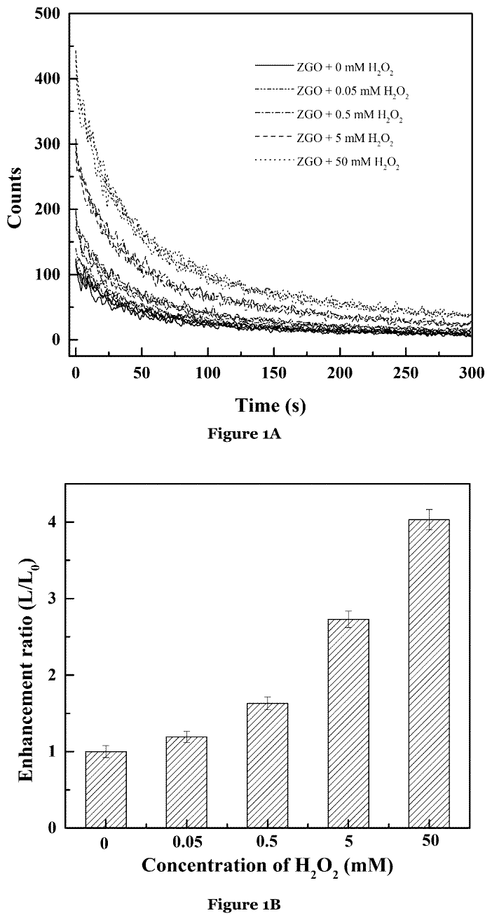

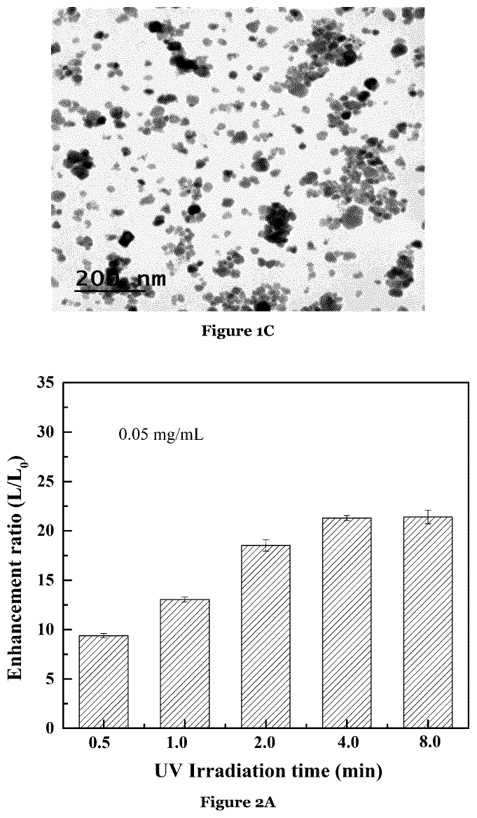

### FIG. 1

FIG. 1A represents the luminescence signal (counts) in function of the time of samples comprising ZGO nanoparticles, ZGO nanoparticles and 0.05 mM H₂O₂, ZGO nanoparticles and 0.5 mM H₂O₂, ZGO nanoparticles and 5 mM H₂O₂, ZGO nanoparticles and 50 mM H₂O₂.

FIG. 1B represents the enhancement ratio of luminescence signal (intensity of the luminescence signal in the presence of H₂O₂/luminescence intensity without H₂O₂) as function of the concentration of H₂O₂ (mM) for samples comprising ZGO nanoparticles, ZGO nanoparticles and 0.05 mM H₂O₂, ZGO nanoparticles and 0.5 mM H₂O₂, ZGO nanoparticles and 5 mM H₂O₂, ZGO nanoparticles and 50 mM H₂O₂.

FIG. 1C represents a transmission electronic microscopy (TEM) image of the ZGO nanoparticles

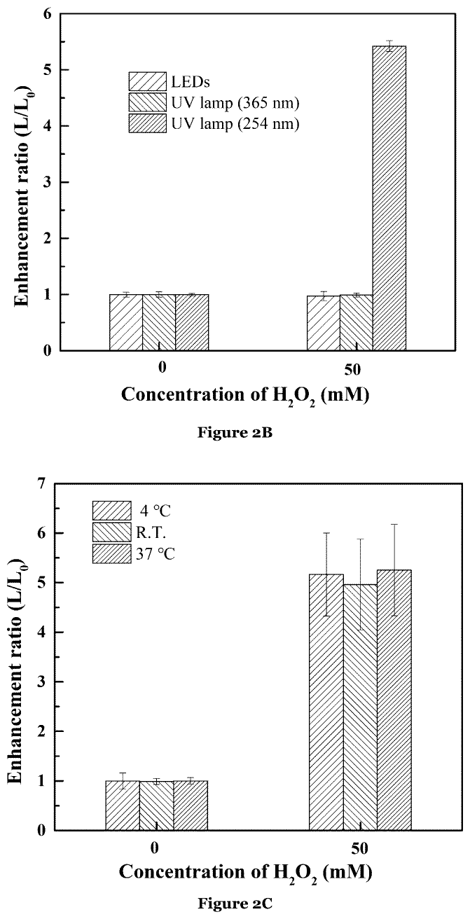

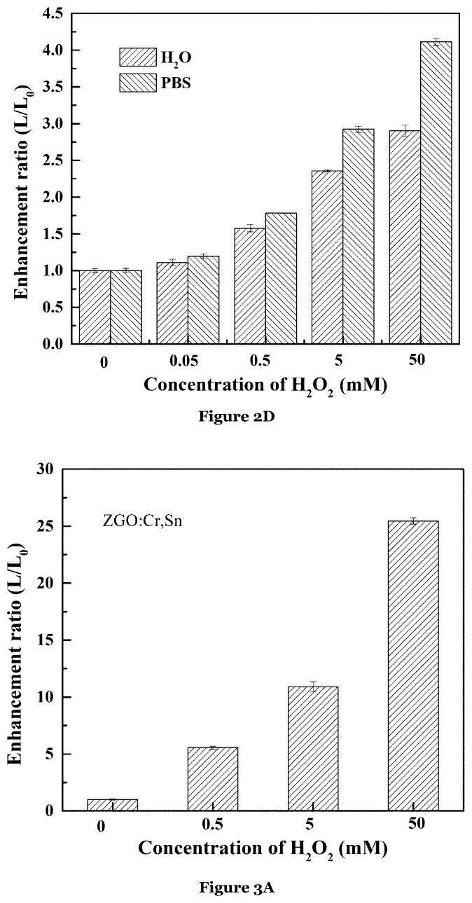

### FIG. 2

FIG. 2A represents the enhancement ratio of luminescence signal as function of the UV irradiation time (min) for samples comprising ZGO nanoparticles and H₂O₂ irradiated for 0.5 minute, 1 minute, 2 minutes, 4 minutes, 8 minutes.

FIG. 2B represents the enhancement ratio of luminescence signal as function of the concentration of H₂O₂ for samples comprising ZGO nanoparticle without H₂O₂ or with 50 mM H₂O₂ and excited with a LED, a UV lamp at 365 nm or a UV lamp at 254 nm.

FIG. 2C represents the enhancement ratio of luminescence signal as function of the concentration of H₂O₂ for samples comprising ZGO nanoparticles and ZGO nanoparticles with 50 mM H₂O₂ at 4 °C, at room temperature or 37 °C.

FIG. 2D represents the enhancement ratio of luminescence signal as function of the concentration of H₂O₂ for samples comprising ZGO nanoparticles, ZGO nanoparticles with 0.05 mM H₂O₂, 0.5 mM H₂O₂, 5 mM H₂O₂, 50 mM H2O2 in a medium comprising H₂O of PBS.

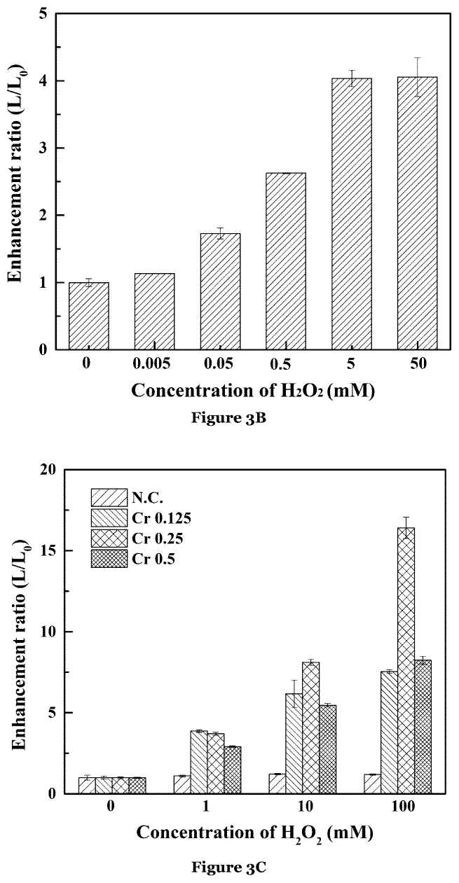

### FIG. 3

FIG. 3A represents the enhancement ratio of luminescence signal as function of the concentration of H₂O₂ for samples comprising ZGO: Cr, Sn nanoparticles, ZGO: Cr, Sn nanoparticles with 0.5 mM H₂O₂, ZGO: Cr, Sn nanoparticles with 5 mM H₂O₂, ZGO: Cr, Sn nanoparticles with 50 mM H₂O₂.

FIG. 3B represents the enhancement ratio of luminescence signal as function of the concentration of H₂O₂ for samples comprising non-calcined ZGO:Cr³⁺ nanoparticles, non-calcined ZGO:Cr³⁺ nanoparticles with 0.005 mM H₂O₂, non-calcined ZGO:Cr³⁺ nanoparticles with 0.05 mM H₂O₂, non-calcined ZGO:Cr³⁺ nanoparticles with 0.5 mM H₂O₂, non-calcined ZGO:Cr³⁺ nanoparticles with 5 mM H₂O₂, non-calcined ZGO:Cr³⁺ nanoparticles with 50 mM H₂O₂.

FIG. 3C represents the enhancement ratio of luminescence signal as function of the concentration of H₂O₂ for samples comprising ZGO nanoparticles, ZGO nanoparticles with 1 mM H₂O₂, ZGO nanoparticles with 10 mM H₂O₂, ZGO nanoparticles with 100 mM H₂O₂.and with different concentrations of Cr ³⁺(0, 0.125%, 0.25%, 0.5%).

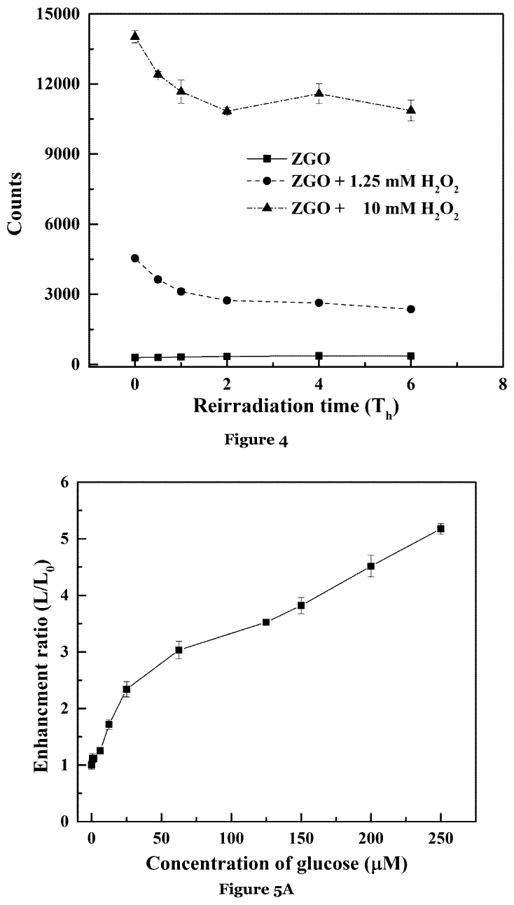

### FIG. 4

FIG. 4 represents the luminescence signal (counts) in function of the number of UV excitation of ZGO nanoparticles, ZGO nanoparticles with 1.25 mM H₂O₂, and ZGO nanoparticles in with 10 mM H₂O₂. Reirradiation of each sample for 2 min with UV is realized after 30 min, 1 h, 2 h, 4 h and 6 h incubation with H2O2.

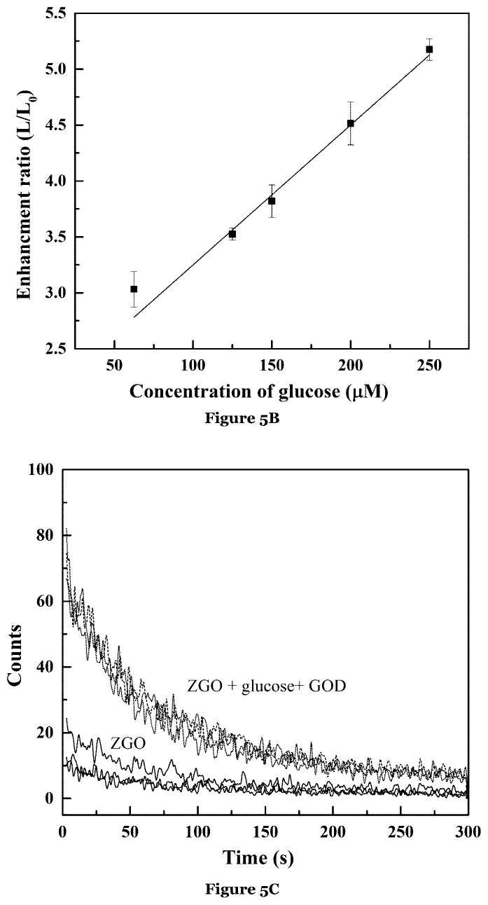

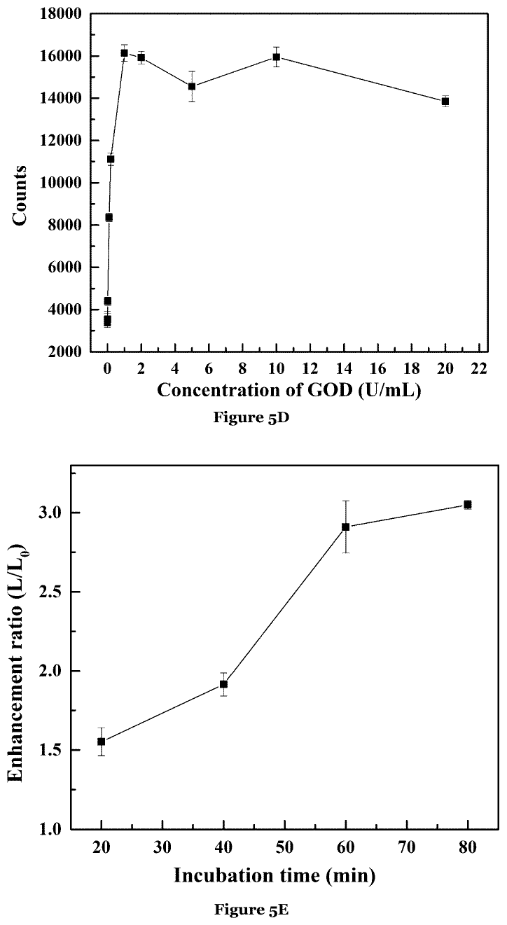

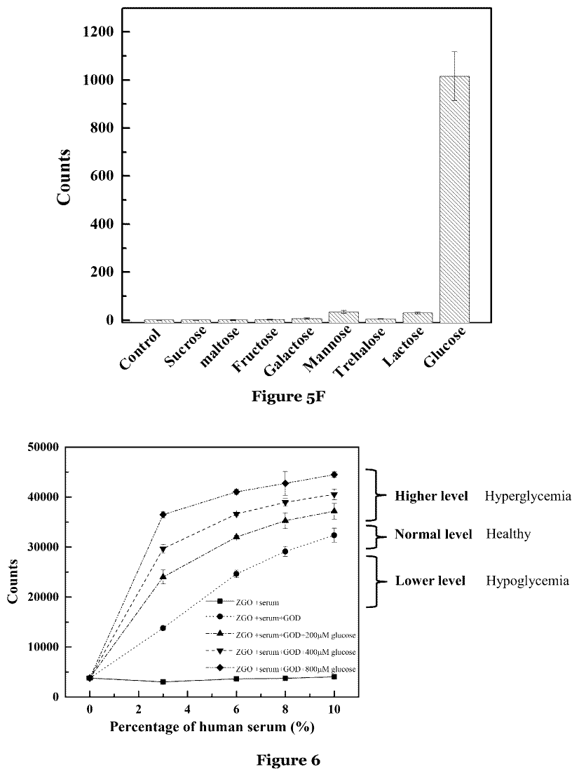

### FIG. 5

FIG. 5A represents the enhancement ratio of luminescence signal as function of the concentration of glucose (μM).

FIG. 5B represents the linearity area of the enhancement ratio of luminescence signal as function of the concentration of glucose (μM).

FIG. 5C represents the luminescence signal (counts) in function of time (second) for a sample comprising ZGO nanoparticles, and ZGO nanoparticles, glucose and glucose oxidase (GOD).

FIG. 5D represents the luminescence signal (counts) in function of the concentration of glucose oxidase (U/ml).

FIG. 5E represents the enhancement ratio of luminescence signal as function of the incubation time.

FIG. 5F represents the luminescence signal (counts) as function of sugar added to GOD: control, sucrose, maltose, fructose, galactose, mannose, xylose, lactose and glucose. The signal amplification is only observed when using glucose.

### FIG. 6

FIG. 6 represents the luminescence signal (counts) in function of the percentage of human serum for samples of ZGO nanoparticles and human serum, ZGO nanoparticles and human serum and glucose oxidase, ZGO nanoparticles and human serum and glucose oxidase and 200 μM glucose, ZGO nanoparticles and human serum and glucose oxidase and 400 μM glucose, ZGO nanoparticles and human serum and glucose oxidase and 800 μM glucose.

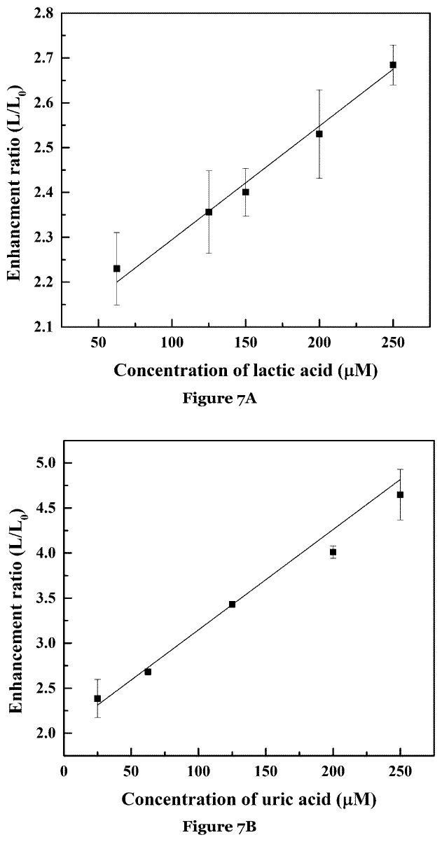

### FIG. 7

FIG. 7A represents the linearity area of the enhancement ratio of luminescence signal as function of the concentration of lactic acid (μM).

FIG. 7B represents the linearity area of the enhancement ratio of luminescence signal as function of the concentration of uric acid (μM).

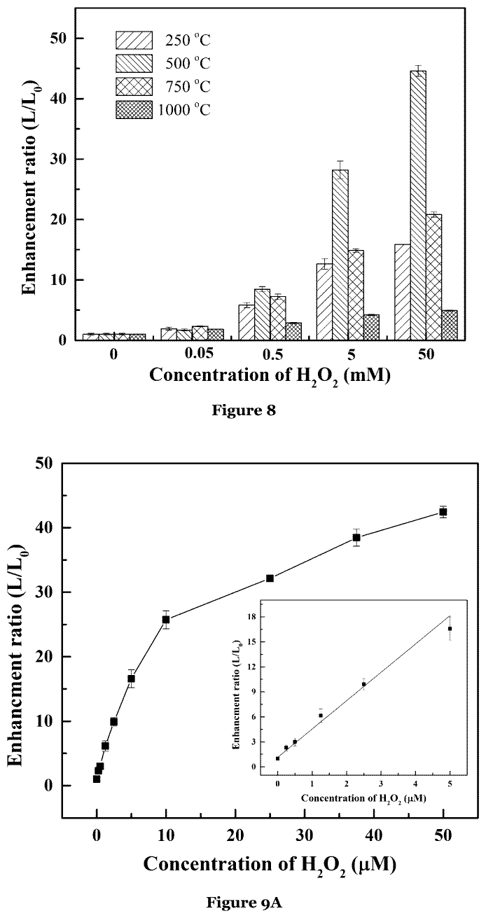

### FIG. 8

FIG. 8 represents the enhancement ratio of luminescence signal as function of the concentration of H₂O₂ for samples comprising ZGO nanoparticles calcined at 250 °C, ZGO nanoparticles calcined at 500 °C, ZGO nanoparticles calcined at 750 °C, ZGO nanoparticles calcined at 1000 °C without H₂O₂ or in the presence of H₂O₂ at a concentration of 0.05 mM, 0.5 mM, 5 mM and 50 mM.

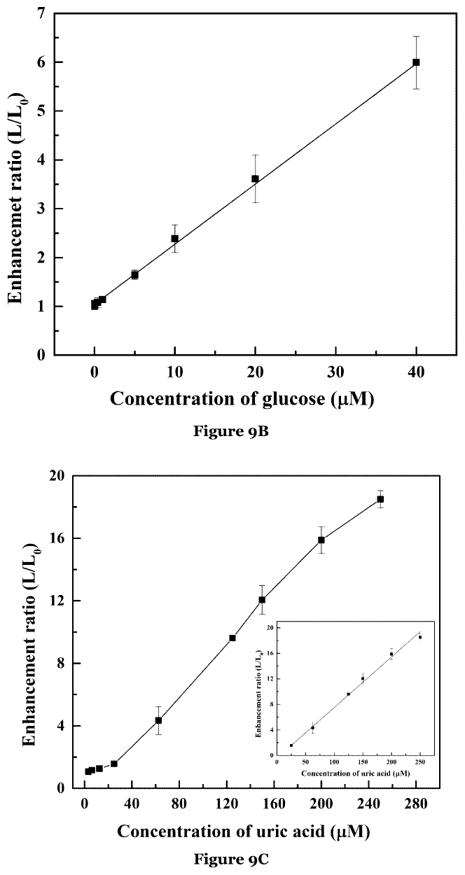

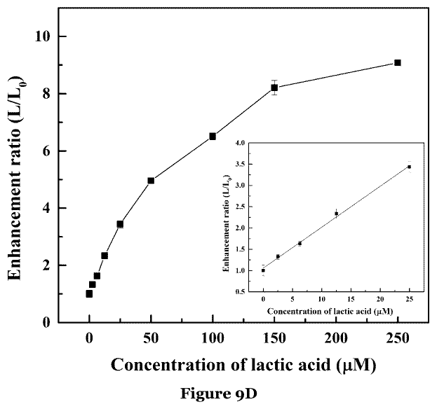

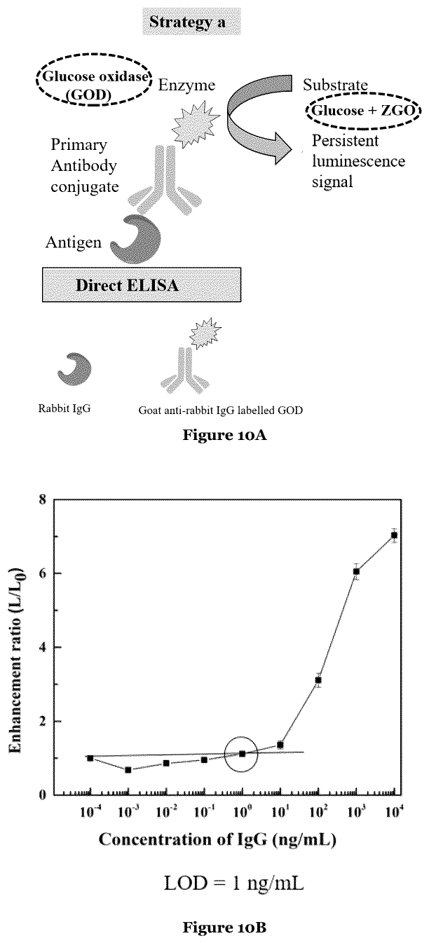

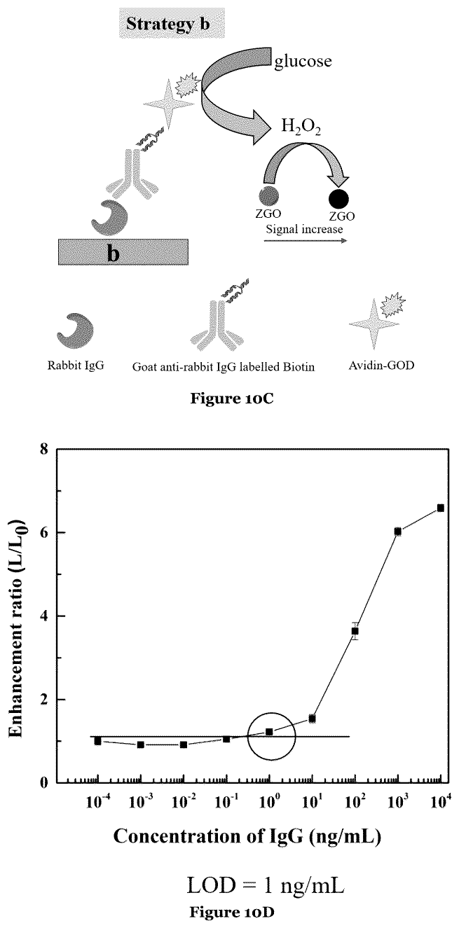

### FIG. 9

FIG. 9A represents the enhancement ratio of luminescence signal as function of the concentration of H₂O₂ for samples comprising ZGO nanoparticles calcined at 500 °C without H₂O₂ and with 0.312 μM H₂O₂, 0.6.25 μM H₂O₂, 1.25 μM H₂O₂, 2.5 μM H₂O₂, 5 μM H₂O₂, 10 μM H₂O₂, 25 μM H₂O₂, 40 μM H₂O₂, 50 μM H₂O₂. The insert is the linearity range.

FIG. 9B represents the linearity range of the enhancement ratio of glucose detection using ZGO nanoparticles calcined at 500 °C.

FIG. 9C represents the enhancement ratio of luminescence signal of ZGO nanoparticles calcined at 500 °C in the presence of different concentrations of uric acid. The insert is the linearity range.

FIG. 9D represents the enhancement ratio of luminescence signal of ZGO nanoparticles calcined at 500 °C in the presence of different concentrations of lactic acid. The insert is the linearity range.

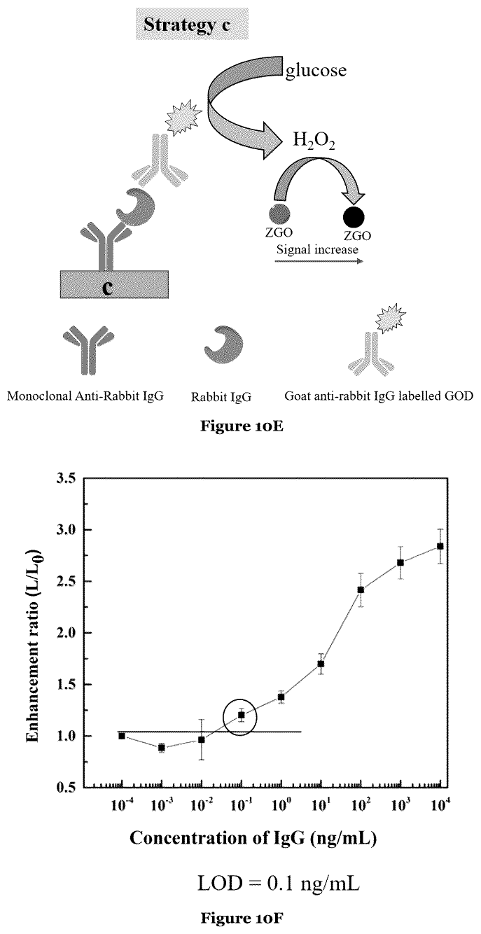

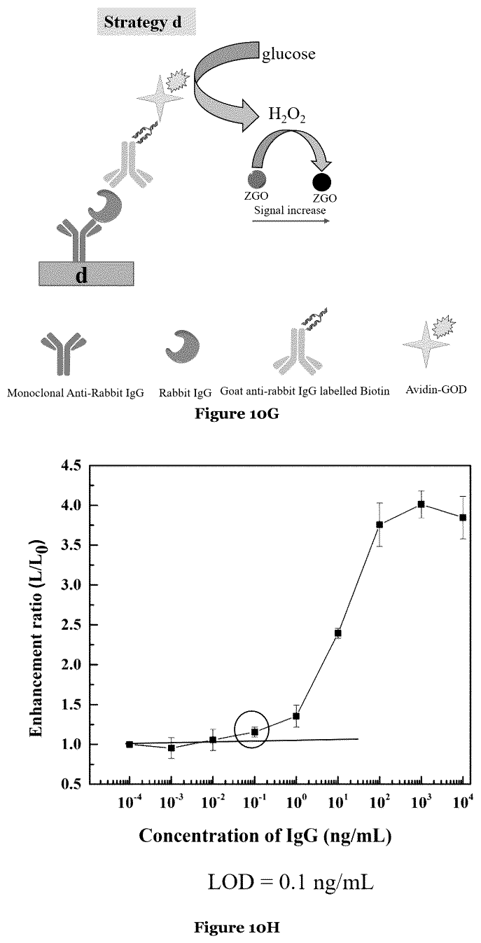

### FIG. 10

FIGS. 10A, 10C, 10E, 10G represent the scheme of antigen using non-functionalized persistent luminescence nanoparticle through an ELISA test.

FIGS. 10B, 10D, 10F and 10H represent the enhancement ratio of luminescence signal as function of the concentration of IgG (ng/ml).

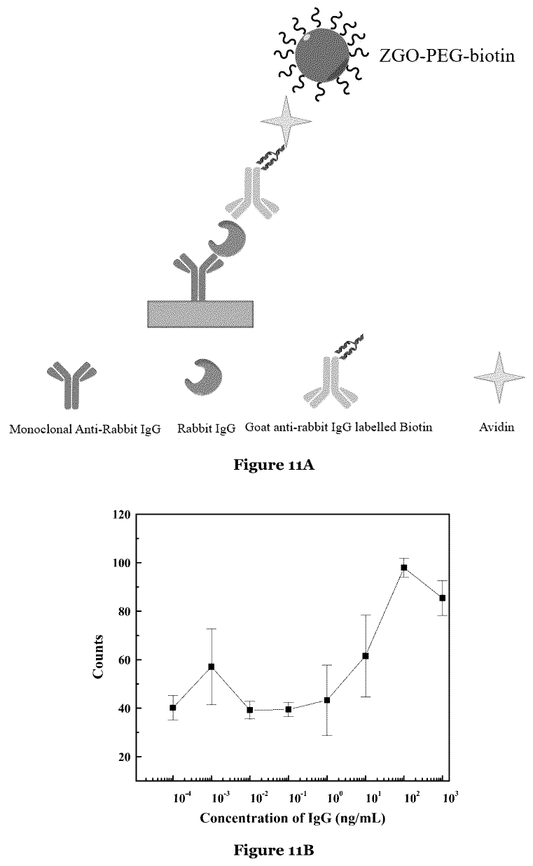

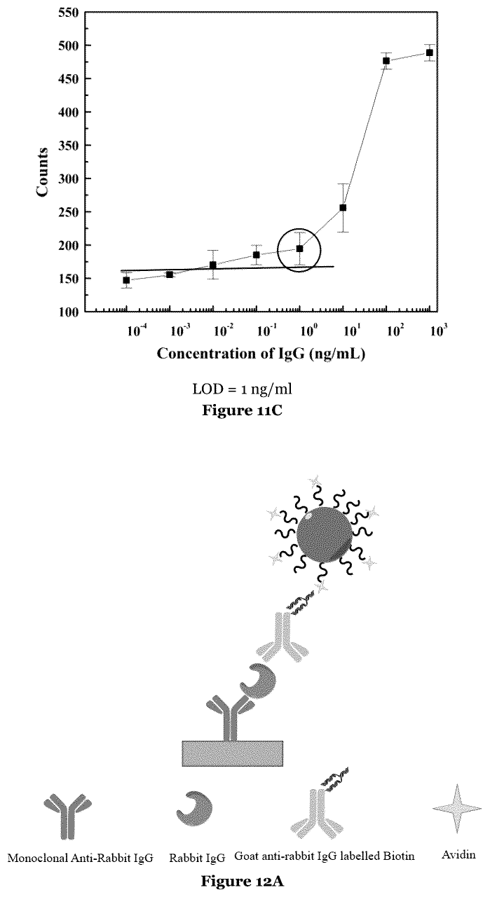

### FIG. 11

FIG. 11A represents the scheme of antigen detection using biotin-functionalized persistent luminescence nanoparticles through an ELISA test.

FIG. 11B represents the luminescence signal (counts) in function of the concentration of IgG (ng/ml) using biotin functionalized ZGO.

FIG. 11C represents the luminescence signal (counts) in function of the concentration of IgG (ng/ml) using biotin functionalized ZGO in presence of H₂O₂.

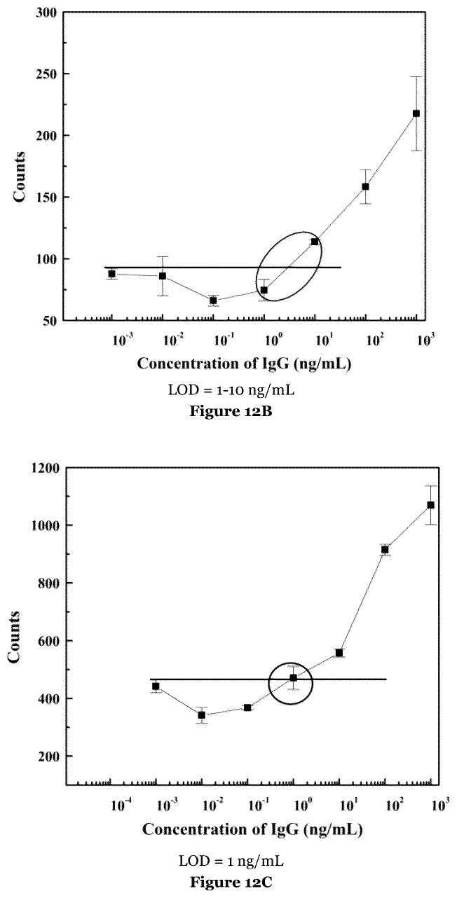

### FIG. 12

FIG. 12A represents the scheme of antigen detection using avidin-functionalized persistent luminescence nanoparticles through an ELISA test.

FIG. 12B represents the luminescence signal (counts) in function of the concentration of IgG (ng/ml) using avidin functionalized ZGO.

FIG. 12C represents the luminescence signal (counts) in function of the concentration of IgG (ng/ml) using avidin functionalized ZGO in presence of H2O2.

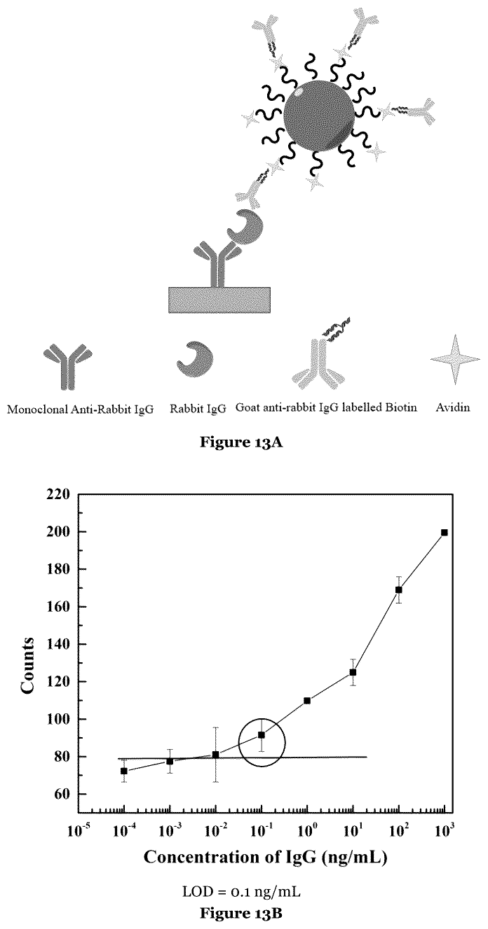

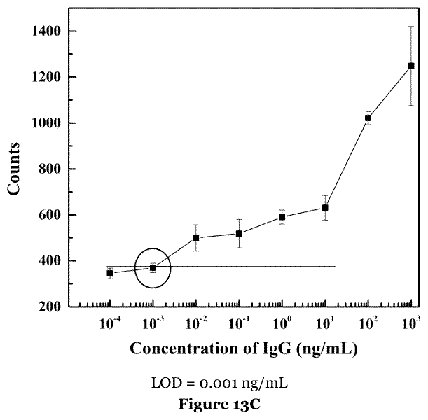

### FIG. 13

FIG. 13A represents the scheme of antigen detection using antibody-functionalized persistent luminescence nanoparticles through an ELISA test.

FIG. 13B represents the luminescence signal (counts) in function of the concentration of IgG (ng/ml) using antibody functionalized ZGO.

FIG. 13C represents the luminescence signal (counts) in function of the concentration of IgG (ng/ml) using antibody functionalized ZGO in presence of H2O2.

### EXAMPLES

### Examples 1: The Inventors Have Evaluated the Effect of the H₂O₂ on the Signal of the Persistent Luminescence

Non-functionalized ZGO persistent luminescence nanoparticle obtained after calcination at 750 °C and dispersed in an aqueous solution at a concentration of 0.025 mg/mL are mixed with different concentrations of H₂O₂:
0 mM H₂O₂;
0.05 mM H₂O₂;
0.5 MM H₂O₂;
5 mM H₂O₂;
50 mM H₂O₂.

After excitation of the mixture for 40 seconds with a UV lamp (254 nm), the signal emitted by the nanoparticles is captured by a photon-counting device (Optima, Biospace lab) for few minutes, typically for 5 min.

The signal emitted by the nanoparticles increases with increasing amount of H₂O₂. The enhancement, defined as the signal of ZGO in the presence of H₂O₂ divided by the signal of ZGO without H₂O₂, is proportional to the amount of H2O2 (see FIGS. 1A and 1B).

FIG. 1C represents a TEM image of the ZGO nanoparticles.

### Example 2: The Inventors Have Evaluated the Influence of Different Parameters on the Signal of the Persistent Luminescence Nanoparticles

Non-functionalized ZGO persistent luminescence nanoparticles obtained after calcination at 750 °C are dispersed in an aqueous solution at a final concentration of 0.025 mg/ml.

### A. Influence of the Irradiation Time

The aqueous suspension comprising the ZGO persistent luminescence nanoparticles is mixed with H₂O₂ at a concentration of 50 mM. The mixture is then excited with a UV lamp at 254 nm for:
0.5 minute;
1 minute;
2 minutes;
4 minutes;
8 minutes.

The signal emitted by the nanoparticles is captured by a photon-counting device (Optima, Biospace lab).

The signal emitted by the persistent luminescence nanoparticles increases when the UV irradiation time increases until 4 minutes of irradiation (see FIG. 2A).

### B. Influence of the Excitation Source

The aqueous suspension comprising the ZGO persistent luminescence nanoparticles is mixed with H₂O₂ at a concentration of 50 mM and is then excited with:
a UV lamp at 254 nm;
a UV lamp at 365 nm;
a LED.

The same protocol is performed for comparison with a mixture of ZGO persistent luminescence nanoparticle without H₂O₂. The signal emitted by the nanoparticles is captured by a photon-counting device (Optima, Biospace lab).

The signal emitted by the nanoparticles without H₂O₂ is low regardless the source of excitation. The signal emitted by the nanoparticles comprising H₂O₂ with a UV lamp at 365 nm or with a LED is comparable to the signal obtained for the mixtures that do not comprise H₂O₂. The signal emitted by the nanoparticles drastically increases when the mixture is mixed with 50 mM of H₂O₂ and is excited with a UV lamp at 254 nm (see FIG. 2B).

### C. Influence of the Temperature

The aqueous suspension comprising the ZGO persistent luminescence nanoparticles is mixed with H₂O₂ at a concentration of 50 mM and is excited with a UV lamp at 254 nm at:
4 °C;
Room temperature (20 °C);
37 °C.

The same protocol is performed with a mixture which does not comprise H₂O₂. The signal emitted by the nanoparticles is captured by a photon-counting device (Optima, Biospace lab). The signal emitted by the nanoparticles without H₂O₂ is low regardless the temperature. The signal emitted by the nanoparticles in the mixture comprising H₂O₂ drastically increases regardless the temperature (see FIG. 2C).

### D. Influence of the Medium

Non-functionalized ZGO persistent luminescence nanoparticle obtained after calcination at 750 °C are dispersed in an aqueous solution comprising H₂O or in PBS at a concentration of 0.05 mg/ml. The suspension is then mixed with different concentrations of H₂O₂:
0 mM H₂O₂ (control);
0.05 mM H₂O₂;
5 mM H₂O₂;
50 mM H₂O₂.

After excitation of the mixture with a UV lamp (254 nm), the signal emitted by the nanoparticles is captured by a photon-counting device (Optima, Biospace lab).

It can be seen that signal emitted by the nanoparticles increase with increasing amount of H₂O₂. Furthermore, the signal is enhanced when the nanoparticles are dispersed in H₂O and in PBS (see FIG. 2D).

### Example 3: The Inventors Have Tested Different Type of Persistent Luminescence Nanoparticles

### A. ZGO:Cr,Sn Persistent Luminescence Nanoparticle

Non-functionalized ZGO persistent luminescence nanoparticle doped with Cr³⁺and Sn²⁺are obtained after calcination at 750 °C. The nanoparticles are dispersed in an aqueous solution at a concentration of 0.05 mg/ml. The suspension is then mixed with various concentration of H₂O₂:
0 mM H₂O₂ (control)
0.5 MM H₂O₂;
5 mM H₂O₂;
50 mM H₂O₂.

After excitation of the mixture with a UV lamp (254 nm), the signal emitted by the nanoparticles is captured by a photon-counting device (Optima, Biospace lab).

The signal emitted by the nanoparticles increases with increasing amount of H₂O₂. The enhancement, defined as the signal of ZGO in the presence of H₂O₂ divided by the signal of ZGO without H₂O₂, is proportional to the amount of H2O2 (see FIG. 3A).

### B. Non-Calcined ZGO:Cr Persistent Luminescence Nanoparticle

Non-functionalized ZGO persistent luminescence nanoparticle doped with Cr³⁺are obtained without calcination. The nanoparticles are dispersed in an aqueous solution at a concentration of 0.05 mg/ml. The suspension is then mixed with various concentrations of H₂O₂:
0 mM H₂O₂ (control);
0.005 mM H2O2;
0.05 mM H2O2;
0.5 mM H₂O₂;
5 mM H₂O₂;
50 mM H₂O₂,

After excitation of the mixture with a UV lamp (254 nm), the signal emitted by the nanoparticles is captured by a photon-counting device (Optima, Biospace lab).

The signal emitted by the nanoparticles increases with increasing amounts of H₂O₂. The enhancement, defined as the signal of ZGO in the presence of H₂O₂ divided by the signal of ZGO without H₂O₂, is proportional to the amount of H2O2 (see FIG. 3B).

### C. ZGO Persistent Luminescence Nanoparticle With Various Concentration of Cr³⁺

Non-functionalized ZGO persistent luminescence nanoparticle without Cr³⁺ and doped with Cr³⁺ at various amount (0.125%, 0.25%, 0.5%) are obtained after calcination at 750 °C.

The nanoparticles are dispersed in an aqueous solution at a concentration of 0.05 mg/ml. The suspension is then mixed with various concentrations of H₂O₂:
0 mM H₂O₂ (control);
1 mM H₂O₂;
10 mM H₂O₂;
100 mM H₂O₂.

After excitation of the mixture for 40 s with a UV lamp (254 nm), the signal emitted by the nanoparticles is captured by a photon-counting device (Optima, Biospace lab).

The signal emitted by the nanoparticles without H₂O₂ (control) is low. The signal emitted by the nanoparticles ZGO without Cr³⁺ is low and comparable to the signal emitted by the nanoparticles without H₂O₂ (control) regardless the concentration of H₂O₂.

The signal emitted by the nanoparticles increase for the nanoparticles doped with Cr³⁺ with increasing amounts of H₂O₂. Best results are obtained using 0.25% of Cr ³⁺ (see FIG. 3C).

### Example 4: The Inventors Have Evaluated the Effect of Several Excitations on the Signal of the Persistent Luminescence Nanoparticle

Non-functionalized ZGO persistent luminescence nanoparticle obtained after calcination at 750 °C and dispersed in an aqueous solution at a concentration of 0.025 mg/mL are mixed with different concentrations of H₂O₂:
0 mM H₂O₂;
1.25 mM H₂O₂;
10 mM H₂O₂.

After excitation of the mixture for 40 s with a UV lamp (254 nm), the signal emitted by the nanoparticles is captured by a photon-counting device (Optima, Biospace lab).

The signal emitted by the nanoparticles increases with increasing amount of H₂O₂ (see FIG. 4). The signal decreases after each excitation probably due to H₂O₂ consumption with UV excitation source (see FIG. 4). However, the signal is still higher than the signal of ZGO alone (without H₂O₂).

### Example 5: The Inventors Have Applied the Method of the Invention to the Detection of Glucose

A. In a 96-well microplate, 25 μL of glucose oxidase (GOD) (1 U/mL), 50 μL of ZGO nanoparticles and 25 μL of glucose were added into each well. After incubation for 1 h at 37 °C to produce H₂O₂, the signal is measured by a photon-counting device (Optima, Biospace lab). The signal emitted by the nanoparticles increases in the presence of GOD and glucose (see FIG. 5C).
B. The same procedure has been performed using different concentrations of glucose (see FIGS. 5A and 5B). The signal emitted by the nanoparticles increases with increasing amounts of glucose. The minimum amount of detectable glucose is 35.8 μM.
C. The same procedure has been tested by varying the concentration of glucose oxidase (GOD) and the incubation time. The signal emitted by the nanoparticles increases with increasing concentration of GOD (see FIG. 5D).

The signal emitted by the nanoparticles increases with increasing incubation time (see FIG. 5E).
D. The same procedure has been performed by replacing glucose in the well microplates by: Nothing (control)
Sucrose
Maltose
Fructose
Galactose
Mannose
Trehalose
Lactose

The signal emitted by the nanoparticles increases in the presence of glucose. The detection is specific to glucose (FIG. 5F).
E. The inventors have also tested the measurement of the glucose in a human serum medium. The signal emitted by the nanoparticles increases in the presence of GOD. In addition, the signal emitted by the nanoparticles increases with increasing percentage of human serum and increasing amount of glucose (see FIG. 6).

### Example 6: The Inventors Have Applied the Method of the Invention to the Detection of Lactic Acid and Uric Acid

A. In a 96-well microplate, 25 μL of lactate oxidase (1 U/mL), 50 μL of ZGO nanoparticles and increasing concentration of lactic acid were added into each well. After incubation for 1 h at 37 °C to produce H₂O₂, the signal is measured by a photon-counting device (Optima, Biospace lab).

The signal emitted by the nanoparticles increases with increasing amount of lactic acid (see FIG. 7A). The minimum amount of detectable lactic acid is 35.3 μM.
B. In a 96-well microplate, 25 μL of uricase (1 U/mL), 50 μL of ZGO nanoparticles and increasing concentration of uric acid were added into each well. After incubation for 1 h at 37 °C to produce H₂O₂, the signal is measured by a photon-counting device (Optima, Biospace lab).

The signal emitted by the nanoparticles increases with increasing amount of uric acid (see FIG. 7B). The minimum amount of detectable uric acid is 20.07 μM.

### Example 7: The Inventors Have Evaluated the Influence of the Temperature of Synthesis of the Persistent Luminescence Nanoparticle

Non-functionalized ZGO persistent luminescence nanoparticle obtained after calcination at various temperature:
250 °C;
500 °C;
750 °C;
1000 °C;
   and dispersed in an aqueous solution at a concentration of 0.025 mg/ml are mixed with different concentrations of H₂O₂:
0 mM H₂O₂;
0.05 mM H₂O₂;
0.5 mM H₂O₂;
5 mM H₂O₂;
50 mM H₂O₂.

After excitation of the mixture with a UV lamp (254 nm), the signal emitted by the nanoparticles is captured by a photon-counting device (Optima, Biospace lab).

The signal emitted by the nanoparticles increases with increasing amount of H₂O₂. Best results are obtained with nanoparticles calcined at 500 °C (see FIG. 8).

### Example 8: The Inventors Have Evaluated the Signal Emitted by Persistent Luminescence Nanoparticles Calcined at 500 °C in Different Conditions

A. Non-functionalized ZGO persistent luminescence nanoparticle obtained after calcination at 500 °C and dispersed in an aqueous solution at a concentration of 0.025 mg/mL are mixed with different concentrations of H₂O₂:
0 mM H₂O₂;
10 mM H₂O₂;
20 mM H₂O₂;
30 mM H₂O₂;
40 mM H₂O₂;
50 mM H₂O₂.

After excitation of the mixture with a UV lamp (254 nm), the signal emitted by the nanoparticles is captured by a photon-counting device (Optima, Biospace lab).

The signal emitted by the nanoparticles increases with increasing amount of H₂O₂. The enhancement, defined as the signal of ZGO in the presence of H₂O₂ divided by the signal of ZGO without H₂O₂, is proportional to the amount of H2O2 (see FIG. 9A).

The minimum amount of detectable H₂O₂ is 0.13 μM.
B. In a 96-well microplate, 25 μL of glucose oxidase (GOD) (1 U/mL), 50 μL of ZGO nanoparticles and increasing concentrations of glucose were added into each well. After incubation for 1 h at 37 °C to produce H₂O₂, the signal is measured by a photon-counting device (Optima, Biospace lab).

The signal emitted by the nanoparticles increases in the presence of glucose (see FIG. 9B). in particular, the signal emitted by the nanoparticles increases with increasing amounts of glucose. The minimum amount of detectable glucose is 0.21 μM.
C. In a 96-well microplate, 25 μL of uricase (1 U/mL), 50 μL of ZGO nanoparticles and increasing concentration of uric acid were added into each well. After incubation for 1 h at 37 °C to produce H₂O₂, the signal is measured by a photon-counting device (Optima, Biospace lab).

The signal emitted by the nanoparticles increases with increasing amount of uric acid (see FIG. 9C). The minimum amount of detectable uric acid is 0.26 μM.
D. In a 96-well microplate, 25 μL of lactate oxidase (1 U/mL), 50 μL of ZGO nanoparticles and increasing concentration of lactic acid were added into each well. After incubation for 1 h at 37 °C to produce H₂O₂, the signal is measured by a photon-counting device (Optima, Biospace lab).

The signal emitted by the nanoparticles increases with increasing amount of lactic acid (see FIG. 9D). The minimum amount of detectable lactic acid is 0.99 μM.

The lowest concentration of molecule that can be detected is lower when persistent luminescence nanoparticles calcined at 500 °C are used compared to the persistent luminescence nanoparticles calcined at 750 °C.

### Example 9: The Inventors Have Elaborated Different ELISA Strategies Using non-Functionalized Persistent Luminescence Nanoparticle for the Detection of Antigen (IgG)

A. 100 μL of different concentration of rabbit IgG in 50 mM carbonate-bicarbonate buffer are incubated overnight at 4 °C. After washing three times with PBS+Tween 20, BSA is used to block the excess sites of the wells and incubated at 37 °C for 60 min. After washing three times, 100 μL of goat anti-rabbit IgG labelled with GOD is added into the plate and incubated at 37 °C for 60 min. After washing three times, 50 μL of glucose (100 mM) and 50 μL of ZGO (0.1 mg/ml) are added into the well of the plate and incubated at 37 °C for 60 min. The signal is measured by a photon-counting device (Optima, Biospace lab) (see the procedure scheme of FIG. 10A).

The minimum amount of detectable antigen is 1 ng/ml (see FIG. 10B).
B. Another procedure has been tested according to the procedure scheme of FIG. 10C. As before, different concentration of Rabbit IgG are incubated overnight at 4 °C in a 96-wells palate. After washes and incubation with BSA, Goat anti-rabbit IgG labelled with biotin is incubated at 37 °C for 1 h. After washing, avidin-GOD is added into the plate and incubated for 1 h. After washing, 50 μL of glucose (100 mM) and 50 μL of ZGO (0.1 mg/ml) are added into the well of the plate and incubated for 1 h. The signal is measured by a photon-counting device (Optima, Biospace lab) (see the procedure scheme of FIG. 10C).

The minimum amount of detectable antigen is 1 ng/ml (see FIG. 10D).
C. Another procedure for the detection of antigen has been tested according to the procedure scheme of FIG. 10 E.

Monoclonal anti-rabbit IgG is first bound to the microplates and after washing and saturation with BSA, rabbi IgG is incubated for 1 h. After washing, goat anti-rabbit IgG labelled with GOD is added into the plate and incubated. 50 μL of glucose (100 mM) and 50 μL of ZGO (0.1 mg/ml) are added into the well of the plate and incubated. The signal is measured by a photon-counting device (Optima, Biospace lab) (see the procedure scheme of FIG. 10E).

The minimum amount of detectable antigen is 0.1 ng/ml (see FIG. 10F).
D. Another procedure for the detection of antigen has been tested according to the procedure scheme of FIG. 10G.

Monoclonal anti-rabbit IgG is bound to the microplates and to the rabbi IgG is incubated. Goat anti-rabbit IgG labelled with biotin is added to the plate and after 1 h avidin-GOD is added into the plate and incubated. 50 μL of glucose (100 mM) and 50 μL of ZGO (0.1 mg/ml) are added into the well of the plate and incubated. The signal is measured by a photon-counting device (Optima, Biospace lab) (see the procedure scheme of FIG. 10G).

The minimum amount of detectable antigen is 0.1 ng/ml (see FIG. 10H).

### Example 10: The Inventors Have Evaluated the Effect of Persistent Luminescence Nanoparticles, Differently Functionalized, for Antigen Detection

A. Monoclonal anti-rabbit IgG is bound to the microplates and incubated with rabbit IgG. Goat anti-rabbit IgG labelled with avidin is then added and finally biotinylated-ZGO nanoparticles (FIG. 11A). The signal of the ZGO nanoparticle is measured by a photon-counting device (Optima, Biospace lab).

The minimum amount of detectable antigen is >10 ng/ml (see FIG. 11B).

The same procedure is repeated in presence of H₂O₂. The minimum amount of detectable antigen is 1 ng/ml (FIG. 11C).
B. Monoclonal anti-rabbit IgG is bound to the microplates and incubated with rabbit IgG. Goat anti-rabbit IgG labelled with biotin is added and incubated with avidin-ZGO nanoparticles (FIG. 12A). The signal of the ZGO nanoparticle is measured by a photon-counting device (Optima, Biospace lab).

The minimum amount of detectable antigen is between 1-10 ng/ml (see FIG. 12B).

The same procedure is repeated in presence of H₂O₂. The minimum amount of detectable antigen is 1 ng/ml (FIG. 12C).
C. Monoclonal anti-rabbit IgG is bound to the microplates and incubated with rabbit IgG. Goat anti-rabbit IgG functionalized-ZGO nanoparticles are then added (FIG. 13A). The signal of the ZGO nanoparticle is measured by a photon-counting device (Optima, Biospace lab). The minimum amount of detectable antigen is 0.1 ng/ml (see FIG. 13B). The same procedure is repeated in presence of H₂O₂. The minimum amount of detectable antigen is 0.001 ng/ml (FIG. 13C).

1. A method for detection or quantification of H₂O₂ in a sample, comprising:
adding a persistent luminescence nanoparticle to the sample, and
measuring an intensity of luminescence of the persistent luminescence nanoparticle,
wherein the presence of H₂O₂ induces an increase of the intensity of luminescence of the persistent luminescence nanoparticle.

2. (canceled)

3. The method according to claim 1, wherein the persistent luminescence nanoparticle is a zinc gallate nanoparticle.

4. The method according to claim 1, wherein the persistent luminescence nanoparticle is ZnGa₂O_{4.}

5. The method according to claim 1, wherein the persistent luminescence nanoparticle is doped with one or more element, optionally co-doped with one or more cation.

6. The method according to claim 1, wherein the persistent luminescence nanoparticle emits light after being excited by an excitation source.

7. The method according to claim 6, wherein the persistent luminescence nanoparticle emits light for at least 1 minute after the excitation is stopped.

8. The method according to claim 6, wherein the excitation source comprises a UV light source, a visible light source, an infrared light source or an X-rays source.

9. The method according to claim 1, wherein the persistent luminescence nanoparticle emits photons at wavelengths between 100 and 1700 nm.

10. The method according to claim 1. further comprising detecting a compound which chemical modification leads to H₂O₂ production in situ or an enzyme which reaction leads to H₂O₂ production in situ, or an analyte which can be associated with a reaction inducing the production of H₂O₂.

11. The method according to claim 10, wherein the enzyme capable of producing H₂O₂ in situ is an oxidase or a superoxide dismutase.

12. The method according to claim 1, wherein the persistent luminescence nanoparticle comprises a coating.

13. The method according to claim 11, wherein the coating is a polymer coating.

14. The method according to claim 1, wherein the persistent luminescence nanoparticle is functionalized by grafting a biological or chemical substance.

15. The method according to claim 1, wherein the persistent luminescence nanoparticle is prepared by a method comprising a step of calcination at a temperature inferior to 750 °C.

16. (canceled)

17. A kit for detecting H₂O₂ in a sample comprising a persistent luminescence nanoparticle as defined in claim 1 and an enzyme catalysing a reaction producing H₂O₂.

18. The method of claim 11, wherein the oxidase is glucose oxidase, lactate oxidase, alcohol oxidase, cholesterol oxidase, galactose oxidase, oxalate oxidase, glyoxal oxidase, glycollate oxidase, monoamine oxidase, or uricase.
